(19) United States
(12) Patent Application Publication
(10) Pub. No.: US 2015/0299766 A1
(43) Pub. Date: Oct. 22, 2015

(54) AUTOMATION OF BARRIER-BASED PLANT NUCLEIC ACID AND PROTEIN EXTRACTION

(71) Applicant: Dow AgroSciences LLC, Indianapolis, IN (US)

(72) Inventors: Alice Wernicki, Zionsville, IN (US); Lakshmi Sastry-Dent, Avon, IN (US); Delkin Orlando Gonzalez, Portland, OR (US)

(21) Appl. No.: 14/380,584

(22) PCT Filed: Feb. 22, 2013
(86) PCT No.: PCT/US13/27209

§ 371 Date: Aug. 22, 2014

Related U.S. Application Data
(60) Provisional application No. 61/602,105, filed on Feb. 23, 2012.

(51) Int. Cl. C12Q 1/68; G01N 1/40

(52) U.S. Cl. CPC C12Q 1/6806; G01N 1/4005; G01N 1/4077; G01N 2001/4016

(57) ABSTRACT

This invention is related to systems and methods for nucleic acid/protein extraction/isolation from biological samples. In particular, automated barrier-based systems for samples from plant tissues/cells are disclosed. The automated systems and methods disclosed provide reliability and high throughput specifically suitable for nucleic acid/protein extraction/isolation from plant tissues/cells. In some embodiments, the automated systems disclosed can further enhance high throughput capacity for nucleic acid/protein extraction/isolation directly from plant tissue samples.

This application claims a priority based on PCT application PCT/US2013/027209 filed on Feb. 22, 2013 and provisional application 61/602,105 which was filed on Feb. 23, 2012, the entire disclosure of which is hereby incorporated by reference.

### FIELD OF THE INVENTION

This invention is generally related to the field of biomolecule extraction/isolation from biological samples, and more specifically the field of automated systems and methods for barrier-based nucleic acid/protein extraction/isolation from plant tissues/cells.

### BACKGROUND OF THE INVENTION

Efficient and reliable isolation of amplifiable nucleic acid from biological samples is an important topic for modern molecular biology. Unlike nucleic acid extraction/isolation from bacteria or mammalian cells, isolating quality genomic DNA from plants can be difficult due to the presence of cell wall and interference from various inhibitory compounds (for example, phenolics, carbohydrates, humic acid, etc). Many nucleic acid isolation kits are available but they have various degrees of success for extracting nucleic acid from plant cells, including for example MagAttract® (67163, Qiagen, Carlsbad, Calif.), SNARe™ (BP693, Bang's Labs, Fishers, Ind.), and MagBind® (M1027-03, Omega-Biotek) kits. Typically, the quality of DNA extracted from these kits cannot consistently generate amplifiable genomic DNA without further purification. Other kits including the Qiagen's DNeasy 96 Plant® (69181, Carlsbad, Calif.) or Omega-Biotek's E.Z.N.A 96 Plant® (D3485-2, Norcross, Calif.), are widely used, but automation capabilities are limited due to the large number of centrifugation and incubation steps that are required. In addition, extraction/isolation of proteins/polypeptides from plant tissues/cells often encounters similar or different problems. Thus, there remains a need for a reliable methodology which can extract nucleic acids/proteins from plant tissues/cells in high throughput with amplifiable quality/purity.

### SUMMARY OF THE INVENTION

This invention is related to systems and methods for nucleic acid/protein extraction/isolation from biological samples. In particular, automated barrier-based systems for samples from plant tissues/cells are disclosed. The automated systems and methods disclosed provide reliability and high throughput specifically suitable for nucleic acid/protein extraction/isolation from plant tissues/cells. In some embodiments, the automated systems disclosed can further enhance high throughput capacity for nucleic acid/protein extraction/isolation directly from plant tissue samples.

In one aspect, provided is an automated system for nucleic acid extraction/isolation from a sample of plant tissue or plant cells. The system comprises:

(a) at least one barrier-based sample processing apparatus;

(b) a mobility mechanism for transporting the sample processing apparatus;

(c) a plurality of magnetic beads and at least one magnetic field generator;

(d) at least one liquid reagent; and

(e) a mechanism for adding and aspirating the liquid reagent(s).

In one embodiment, the system does not comprise a centrifuge device. In another embodiment, the system does not comprise a shaker for mixing solution with the sample processing apparatus.

In one embodiment, the at least one barrier-based sample processing apparatus comprises:

(i) an input zone for receiving the sample with magnetic particles therein;

(ii) a first reaction zone for receiving a first reagent therein; and

(iii) a second reaction zone downstream of the first reaction zone for receiving a second reagent therein; and

(iv) a force movable between a first position adjacent the input zone and a second position adjacent the second reaction zone; wherein the force urges the fraction-bound solid phase substrate from the input zone and into the first reaction zone; and the force urges the fraction-bound solid phase substrate into the second reaction zone.

In another embodiment, the at least one barrier-based sample processing apparatus comprises:

(i) an input zone for receiving the sample therein;

(ii) a phase-gate zone for receiving an isolation buffer therein;

(iii) an output zone for receiving a reagent therein; and

(iv) a force movable between a first position adjacent the input zone and a second position adjacent the output zone; wherein the force urges the fraction-bound solid phase substrate from the input zone, through the phase-gate zone and into the output zone.

In some embodiments, the phase-gate zone communicates with the input zone. In some embodiments, the output zone communicates with the phase-gate zone.

In another embodiment, the at least one barrier-based sample processing apparatus comprises:

(i) an input zone for receiving the sample therein;

(ii) an output zone for receiving a reagent therein;

(iii) a pathway interconnecting the input zone and the output zone, and

(iv) a force field movable between a first position adjacent the input zone and a second position adjacent the output zone; wherein the force urges the fraction-bound solid phase substrate from the input zone, through the pathway and into the output zone.

In another embodiment, the at least one barrier-based sample processing apparatus comprises:

(i) an input zone for receiving the sample therein;

(ii) a labeling zone for receiving a labeling buffer therein, the labeling buffer including a label that binds to the fraction-bound solid phase substrate to provide a labeled fraction-bound solid phase substrate;

(iii) a reaction zone for receiving a reagent therein, the reagent reacting in response to the label; and (iv) a force field movable between a first position adjacent the input zone and a second position adjacent the reaction zone; wherein the force urges the fraction-bound solid phase substrate from the input zone, through the labeling zone and into the reaction zone; and the reaction of the reagent with the label of the labeled fraction-bound solid phase substrate allows for the quantifying of the level of fraction in the biological sample.

In another embodiment, the at least one barrier-based sample processing apparatus comprises:

(i) an input zone for receiving the sample therein, the fraction binding to the solid phase substrate to form a fraction-bound solid phase substrate;

(ii) a phase-gate zone communicating with the input zone and receiving an isolation buffer therein; an elution zone communicating with the phase-gate zone and receiving a reagent therein, wherein the reagent extracting the fraction from the fraction-bound solid phase substrate;

(iii) a wash zone having an input communicating with the elution zone and an output communicating with the input zone, the wash zone receiving a wash buffer therein for regenerating the solid phase substrate; and

(iv) a force movable between a first position adjacent the input zone, a second position adjacent the phase-gate zone, a third position adjacent the elution zone, and a fourth position adjacent the wash zone; wherein the force sequentially moves from the first position to the second position so as to urge the fraction-bound solid phase substrate from the input zone to the phase-gate zone; from the second position to the third position so as to urge the fraction-bound solid phase substrate from the phase-gate zone to the elution zone wherein the fraction is extracted from the fraction-bound solid phase substrate such that solid phase substrate remains; from the third position to the fourth position so as to urge the solid phase substrate from the elution zone to the wash zone wherein the solid phase substrate is regenerated; and from the fourth position to the first position so as to urge the solid phase substrate from the wash zone to the input zone wherein the solid phase substrate is free to bind with an additional fraction; and optionally

(v) a force movable between a first position adjacent the input zone, a second position adjacent the phase-gate zone, and a third position adjacent the elution zone; wherein the force sequentially moves from the first position to the second position so as to urge the fraction-bound solid phase substrate from the input zone to the phase-gate zone; from the second position to the third position so as to urge the fraction-bound solid phase substrate from the phase-gate zone to the elution zone, from the third position to a position where the force does not affect the movement of the beads such that the liquid in the intersticial space between the beads is released, from a position where the forced does not affect the movement of the beads to the third position, from the third position to the fourth or second position wherein the fraction is extracted from the fraction-bound solid phase substrate such that solid phase substrate remains in the fourth or second position and the eluent is isolated from the solid phase substrate.

In another embodiment, the at least one barrier-based sample processing apparatus comprises a platform comprising:

(i) a channel having an input and an output, the channel adapted for receiving a cell culture therein;

(ii) a solid phase substrate in the channel,

(iii) a biomolecule binding to the solid phase substrate to form a biomolecule-bound solid phase substrate;

(iv) a phase-gate zone having an input communicating with the channel, the phase-gate zone receiving an isolation buffer therein;

(v) an output zone for receiving a reagent therein; and

(vi) a force movable between a first position adjacent the channel and a second position adjacent the output zone; wherein the force urges the biomolecule-bound solid phase substrate from the channel, through the phase-gate zone and into the output zone.

Additional embodiments of the at least one barrier-based sample processing apparatus are also disclosed in WO 2011/106044A1, US 2011/0213133, and US 2011/0212509, the contents of which are herein incorporated by reference in their entireties.

In one embodiment, the at least one barrier-based sample processing apparatus comprises:

(i) a first chamber for receiving the sample comprising a material of interest;

(ii) a second chamber in communication with the first chamber; and

(iii) a lipophilic or immiscible barrier between the first and second chambers;

wherein upon application of an external force to the material of interest, and positional movement of the force relative to the first chamber and the second chamber effects transfer of the material of interest from the sample in the first chamber, into and through the lipophilic or immiscible barrier, and into the second chamber, wherein the material of interest is extracted from the sample which remains in the first chamber.

In another embodiment, the at least one barrier-based sample processing apparatus comprises: (i) two or more sample processing chambers comprising sample processing reagents; and (ii) a water and alcohol immiscible, hydrophobic, or lipophilic material between at least two of said two or more sample processing, such that a sample moves through said material when moved between the first and second processing chambers.

In another embodiment, the at least one barrier-based sample processing apparatus comprises: (i) a first chamber into which a sample comprising a material of interest can be placed; (ii) a second chamber in communication with the first chamber; and (iii) a lipophilic material disposed between the first and second chambers; wherein upon application of an external force to the sample, the material associates with the force, and positional movement of the force relative to the first chamber and the second chamber effects transfer of the material from the sample in the first chamber, into and through the lipophilic material and into the second chamber, wherein the material is extracted from the sample which remains in the first chamber.

In another embodiment, the at least one barrier-based sample processing apparatus comprises: (i) a first chamber comprising magnetic particles capable of associating with an analyte in a sample placed in the first chamber; (ii) a second chamber in communication with the first chamber; a lipophilic material disposed between the first and second chambers; wherein a magnetic force applied to the magnetic particles attracts the magnetic particles and the analyte associated with the magnetic particles, and wherein movement of the magnetic force with respect to the magnetic particles moves the magnetic particles and the associated analyte into and through the lipophilic material and into the second chamber, thereby providing in the second chamber the analyte separated from the sample.

Additional embodiments of the at least one barrier-based sample processing apparatus are also disclosed in WO 2009/111316A2, US 2009/0246782, US 2011/0269190, and US 2011/0306109, the contents of which are herein incorporated by reference in their entireties.

In one embodiment, the at least one barrier-based sample processing apparatus comprises:

(i) an inlet system provided in an area of the first liquid type;

(ii) a first preparation system provided in an area of the first liquid type; and

(iii) a barrier system provided in an area of the second liquid type;

wherein the inlet system and the first preparation system are separated by the barrier system, and wherein the inlet system is adapted to receive a sample comprising a material of interest and the first preparation system is adapted to receive a receiving liquid.

In another embodiment, the at least one barrier-based sample processing apparatus comprises: (A) a first substrate, the first substrate having a first surface comprising at least two area types, (B) a first area type with a first contact angle with water and (C) a second area type with a second contact angle with water, the first contact angle being smaller than the second contact angle, and; (D) a second substrate, the second substrate having a second surface positioned substantially parallel with the first surface at a distance from the first surface of the first substrate; the first surface of the first substrate, or the first surface of the first substrate and the second surface of the second substrate, defines:

(i) an inlet system provided in an area of the first type;

(ii) a first preparation system provided in an area of the first type; and

(iii) a barrier system provided in an area of the second type;

wherein the inlet system and the first preparation system are separated by the barrier system, and wherein the inlet system is adapted to receive a sample liquid comprising the sample and the first preparation system is adapted to receive a receiving liquid.

Additional embodiments of the at least one barrier-based sample processing apparatus are also disclosed in WO 2011/098989A1, the content of which is herein incorporated by reference in its entirety.

In one embodiment, the at least one barrier-based sample processing apparatus uses a nucleic acid separation method comprising:

(i) allowing nucleic acid in the sample to bind to one or more magnetic beads; and

(ii) passing the nucleic acid-magnetic bead complex through a lipophilic or immiscible barrier to separate the nucleic acid from the sample;

wherein the nucleic acid-bead complex is passed through and separated from the immiscible barrier with an applied magnetic field.

In another embodiment, the at least one barrier-based sample processing apparatus uses a nucleic acid separation method comprising: (i) exposing a sample comprising cells containing nucleic acid to an aqueous mixture comprising a lytic reagent and one or more beads capable of binding the nucleic acid released from said cells to form a nucleic acid-bead complex; and (ii) passing the nucleic acid-bead complex through an immiscible liquid layer to separate the nucleic acid from the aqueous mixture, wherein the one or more beads are magnetic, and the nucleic acid-bead complex is passed through and separated from the immiscible liquid layer with an applied magnetic field.

In another embodiment, the at least one barrier-based sample processing apparatus uses a method of transferring nucleic acid comprising: (i) contacting nucleic acid at a first location with one or more beads to form a nucleic acid-bead complex in a liquid; and (ii) transporting the nucleic acid-bead complex to a second location separated from the first location by an intermediary layer, wherein said intermediary layer is immiscible with the liquid, wherein the one or more beads are magnetic, and the nucleic acid-bead complex is passed through and separated from the intermediary layer with an applied magnetic field.

Additional embodiments of the at least one barrier-based sample processing apparatus are also disclosed in U.S. Pat. No. 8,017,340, the content of which is herein incorporated by reference in its entirety.

In one embodiment, the at least one barrier-based sample processing apparatus uses a nucleic acid isolation method comprising: (i) contacting the sample with a solid support in the absence of any chaotropic agent, the solid support comprising an organic polymer, whereby nucleic acid in the sample is bound to the support in a sequence-independent manner in the absence of any chaotropic agent, and (ii) separating the support with bound nucleic acid from the sample.

In another embodiment, the at least one barrier-based sample processing apparatus uses a method of isolating genomic DNA from a sample comprising (i) contacting said sample with a detergent and a solid support in the absence of any chaotropic agent, the solid support comprising an organic polymer, whereby soluble genomic DNA in said sample is bound to the support in a sequence-independent manner in the presence of the detergent and absence of any chaotropic agent, and (ii) separating said support with bound genomic DNA from the sample.

In another embodiment, the at least one barrier-based sample processing apparatus uses a method of isolating RNA and genomic DNA from a sample comprising (i) contacting said sample with a detergent and a solid support in the absence of any chaotropic agent, the solid support comprising an organic polymer, whereby soluble genomic DNA in said sample is bound to the support in a sequence-independent manner in the presence of the detergent and absence of any chaotropic agent; (ii) separating said support with bound genomic DNA from the sample; and (iii) isolating RNA from said sample.

In another embodiment, the at least one barrier-based sample processing apparatus uses a method of isolating genomic DNA from cells in a sample comprising (i) obtaining cells from said sample by immunomagnetic separation; (ii) producing a lysate by contacting said cells with a detergent and a solid support in the absence of any chaotropic agent, the solid support comprising an organic polymer, whereby soluble genomic DNA in said lysate is bound to the support in a sequence-independent manner in the presence of the detergent and absence of any chaotropic agent; and (iii) separating said support with bound genomic DNA from said lysate.

In another embodiment, the at least one barrier-based sample processing apparatus uses a method of isolating RNA and genomic DNA from cells in a sample comprising (i) obtaining cells from said sample by immunomagnetic separation; (ii) producing a lysate by contacting said cells with a detergent and a solid support in the absence of any chaotropic agent, the solid support comprising an organic polymer, whereby soluble genomic DNA in said lysate is bound to the support in a sequence-independent manner in the presence of the detergent and absence of any chaotropic agent; (iii) separating said support with bound genomic DNA from said lysate; and (iv) isolating RNA from said lysate.

In another embodiment, the at least one barrier-based sample processing apparatus uses a method of isolating genomic DNA from a sample containing intact cells comprising (i) contacting said sample containing intact cells with a detergent and magnetic particles, said magnetic particles comprising an organic polymer, and whereby soluble genomic DNA from said sample is bound to the surface of said particles in a sequence-independent manner in the presence of the detergent and absence of any chaotropic agent, and (ii) separating said magnetic particles with bound genomic DNA from the sample.

In another embodiment, the at least one barrier-based sample processing apparatus uses a method of isolating genomic DNA from cells in a sample comprising (i) obtaining cells from said sample by immunomagnetic separation; (ii) producing a lysate by contacting said cells with a detergent and magnetic particles in the absence of any chaotropic agent, the magnetic particles comprising an organic polymer whereby soluble genomic DNA in said lysate is bound to the surface of the support in a sequence-independent manner in the presence of the detergent and absence of any chaotropic agent; and (iii) separating said magnetic particles with bound genomic DNA from said lysate.

In another embodiment, the at least one barrier-based sample processing apparatus uses a method of isolating RNA and genomic DNA from cells in a sample comprising (i) obtaining cells from said sample by immunomagnetic separation; (ii) producing a lysate by contacting said cells with a detergent and magnetic particles in the absence of any chaotropic agent, the magnetic particles comprising an organic polymer, and whereby soluble genomic DNA in said lysate is bound to the surface of the support in a sequence-independent manner in the presence of the detergent and absence of any chaotropic agent; (iii) separating said magnetic particles with bound genomic DNA from said lysate; and (iv) isolating RNA from said lysate.

In another embodiment, the at least one barrier-based sample processing apparatus uses a method of isolating genomic DNA from a sample comprising (i) contacting said sample with a detergent and magnetic particles comprising an organic polymer wherein the surface of the magnetic particle has a negative, neutral or hydrophobic surface, and whereby soluble genomic DNA in said sample is bound to the surface of said particles in a sequence-independent manner; and (ii) separating said magnetic particles with the bound genomic DNA from the sample.

Additional embodiments of the at least one barrier-based sample processing apparatus are also disclosed in U.S. Pat. Nos. 7,173,124 and 7,989,614, the contents of which are herein incorporated by reference in their entireties.

In another embodiment, the mobility mechanism comprises a conveyor or a disk. In a further or alternative embodiment, the mobility mechanism further comprises a robotic arm. In another embodiment, the magnetic field generator comprises magnetic separator pins. In another embodiment, the conveyor comprises a vertical conveyor or a horizontal conveyer. In another embodiment, the magnetic field generator comprises magnetic separator pins or an electromechanical device. In another embodiment, the magnetic field generator comprises a magnetic donut rack, donut magnets, or one or more electromagnetic field generators.

In one embodiment, the at least one liquid reagent comprises a nucleic acid binding solution or an elution solution. In a further or alternative embodiment, the nucleic acid binding solution comprises isopropanol or chaotropic salt. In a further or alternative embodiment, the elution solution comprises Tris-HCl at pH 7.0-9.0. In another embodiment, the elution solution comprises Tris-HCl at about pH 8.5. In another embodiment, the elution solution comprises Tris-HCl at about pH 8.0.

In another embodiment, the mechanism for adding and aspirating the liquid reagent(s) comprises a pipetting station. In a further or alternative embodiment, the mechanism for adding and aspirating the liquid reagent(s) comprises a Vprep® pipetting station available from Agilent Technologies Company, a microplate dispenser such as a Multidrop available from ThermoFisher, or a Evo pipetting station available from Tecan.

In one embodiment, the plant is selected from the group consisting of cotton, canola, corn, soybean, sunflower, or wheat. In another embodiment, the plant tissue comprises a punch of leaf or cotyledon tissue. In a further or alternative embodiment, the punch comprises about a 12 mm diameter tissue. In another embodiment, the plant tissue comprises a punch number from 1 to 100. In a further or alternative embodiment, the punch number is 4 or 8. In one embodiment, the nucleic acid comprises DNA or RNA. In a further or alternative embodiment, the nucleic acid comprises genomic DNA. In another embodiment, the system comprises an Agilent BioCel system. In some embodiments, the automated systems disclosed do not contain a filter device. In some embodiment, the liquid reagent(s) do not contain organic solvents. In some embodiment, the liquid reagent(s) do not contain phenol, chloroform, or isoamyl alcohol. In some embodiments, the liquid reagent(s) do not contain guanidine hydrochloride or guanidine thiocynate.

In one embodiment, the throughput of the system is greater than 650 extractions per hour. In another embodiment, the throughput of the system is equal or greater than 1,000 extractions per hour. In another embodiment, the throughput of the system is equal or greater than 4,000 extractions per hour. In another embodiment, the throughput of the system is between 200 and 3,000 extractions per hour. In another embodiment, the throughput of the system is between 650 and 4,000 extractions per hour.

In some embodiments, the automated systems provided comprise a plurality of electromechanical or magnetic devices. In further or alternative embodiments, the electromechanical device or devices can be turned on or off or can be positioned such that device(s) does or does not affect movement of the magnetic beads. In some embodiments, the nucleic acid binding solution does not contain guanidine hydrochloride or guanidine thiocynate. In some embodiments, the methods described exclude the use of a filter device. In some embodiments, the methods described exclude the use of organic solvents. In some embodiment, the organic solvents excluded are selected from the group consisting of phenol, chloroform, and isoamyl alcohol.

### BRIEF DESCRIPTION OF THE DRAWINGS

FIG. 1 shows an exemplary embodiment of the automated systems provided herein with a conveyor (14). FIG. 1A shows an input area (11), a liquid transfer station (12), at least one barrier-based sample processing apparatus/device, a magnetic working area (13) of the automated system provided, where one or more magnetic forces or magnetic fields (15) can be applied externally. FIG. 1B shows an exemplary embodiment of barrier-based sample processing apparatus/device (19) with an elution well (16), a barrier well (17), and a sample receiving well (18).

FIG. 2 shows another exemplary embodiment of the automated systems provided herein with a conveyor. FIG. 2A shows an exemplary embodiment of an input station (21) housing four barrier-based sample processing apparatuses/devices (22), where each barrier-based sample processing apparatus/device contains an elution well (23), a barrier well (24), and a sample receiving well (25). FIG. 2B shows an input area (26), a liquid transfer station (27), at least one barrier-based sample processing apparatus/device, a magnetic working area (28) of the automated system provided, where one or more magnetic forces or magnetic fields (29) can be applied externally.

FIG. 3 shows an illustration of another exemplary embodiment of the automated systems provided herein with a rotating disk (31). A rotating anchor axis (32) and multiple barrier-based sample processing apparatuses/devices with different sizes (33 and 34) are shown.

FIG. 4 shows an illustration of another exemplary embodiment of the automated systems provided herein with a rotating disk (41) and an aspirating/dispensing station (46). A rotating anchor axis (42) and multiple barrier-based sample processing apparatuses/devices with different sizes (43 and 44) are shown. Also shown are multiple dispensing/aspirating nozzles (45) of the aspirating/dispensing station (46).

FIG. 5 shows a close view of an exemplary aspirating/dispensing station (51) as part of the automated system shown in FIG. 4. A side wall (52) and two liquid transfer needles (52) of the aspirating/dispensing station (51) are shown.

FIG. 6 shows an illustration of another exemplary embodiment of the automated systems provided herein with a rotating disk (61) and an aspirating/dispensing station (62). A rotating anchor axis (63) and a magnet or magnetic field generator (64) for producing a magnetic force or magnetic field are shown.

FIG. 7 shows an exemplary embodiment of a movable input station for at least one barrier-based sample processing apparatus/device. FIG. 7A shows a sled (72) can move along a pair of rails (73) using multiple bushings (71). A magnet or magnetic field generator (74) is also shown. FIG. 7B shows an array of devices (75) with two barrier-based sample processing apparatuses/devices (79), where each barrier-based sample processing apparatus/device contains an elution well (76), a barrier well (77), and a sample receiving well (78).

FIG. 8A shows an exemplary embodiment of a stationary input station, where the magnet or magnetic field generator (83) can move along a pair of rails (82) using a pair of bushings (81) under an input station platform (84). The direction (85) of the movable magnet or magnetic field generator (83) is shown.

FIG. 9 shows another exemplary embodiment of the automated system provided herein. FIG. 9A shows an automated pipetting station or pipetting system with multiple pipette tips (92) for liquid transfer and an automated platform of input station (97) with two barrier-based sample processing apparatuses/devices (96). Also shown are reservoirs (95) for various reagents or solutions, tubes with samples to be processed (94), and tubes with eluted solution from samples that have been process in the system provided (93). FIG. 9B shows the electronic control and feedback relationships between the various components of the automated system provided herein.

FIG. 10 shows another exemplary embodiment of the automated system provided herein. A conveyor (101), an input area (102), a sample process station (103), a magnetic field station (104), and a liquid transfer station (105) are shown.

FIG. 11 shows another exemplary embodiment of the automated system provided herein with a vertical conveyor. FIG. 11A shows a shelf (111) linked to the vertical conveyor for housing multiple barrier-based sample processing apparatuses/devices (112; two are shown). FIG. 11B show that multiple shelves are linked to the vertical conveyor (115). Also shown are multiple liquid transfer stations (114) linked to a tower (113) next to the vertical conveyor (115).

FIG. 12 shows another exemplary embodiment of the automated system provided herein with a horizontal conveyor. FIG. 12A shows top view of a shelf without plate (left panel) and with plate (122; right panel), and a slot (121) of the shelf. FIG. 12B shows a tab (123), a shelf with plate (127), a magnet or magnetic field generator (125), and a belt (124) of the horizontal conveyor. Also shown is the slot (126) of the shelf, and direction of the belt (128).

### DETAILED DESCRIPTION OF THE INVENTION

Plant tissue samples often produce inhibitory compounds that negatively impact the quality of genomic DNA/protein samples and limit their use in many downstream applications. While most commercially available DNA extraction chemistries have been designed to cover a broad spectrum of plants, they have not been proven robust enough to consistently yield amplifiable DNA from various plants including cotton. Currently, the Qiagen DNeasy 96 Plant® kit is often regarded as the “standard” extraction method for plant samples. However, the semi-automated filter-based procedure of DNeasy is costly and labor intensive.

Barrier-based nucleic acid extraction/isolation kits are known and attempts have been made to purify genomic DNA from plant samples using such commercial kits. However, none of these commercial kits are adapted for automation for plant tissues for various reasons. For example, Immiscible Filtration Assisted by Surface Tension (IFAST) is a barrier-based technology allowing rapid processing of samples using existing paramagnetic bead technology (see Berry et al. (2011) Lab Chip 11: 1747-1753 available at world wide web pubs.rsc.org/en/Content/ArticleLanding/2011/LC/c11c00004g; WO 2011/106044A1; US 2011/0213133; and US 2011/0212509, the contents of which are herein incorporated by reference in their entireties). Other barrier-based nucleic acid isolation methods are disclosed in, for example, WO 2009/111316A2, US 2009/0246782, US 2011/0269190, and US 2011/0306109, WO 2011/098989A1, U.S. Pat. Nos. 8,017,340, 7,173,124, and 7,989,614, the contents of which are herein incorporated by reference in their entireties.

Provided are methods and systems for automating the processing of plant samples in barrier-based sample processing apparatuses/devices. In some embodiments, the methods and systems provided utilize a conveyor to move the barrier-based sample processing apparatuses/devices to and from the liquid handling stations and the magnet/magnetic field generator that are required to produce a purified sample.

In addition to high throughput/capacity, automation generally has advantage to reduce multiple user interactions. Automation also enables combination with downstream analysis, for example, PCR amplification or other detection methods. Automation is generally believed to be more economical, efficient, reproducible, and accurate for the processing of samples and assays.

As used herein, the phrase “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. The phrases “nucleic acid” or “nucleic acid sequence” may also be used interchangeably with gene, cDNA, and mRNA encoded by a gene. In some embodiment, the nucleic acid molecule is a segment of DNA. Nucleotides are indicated by their bases by the standard abbreviations, for example: adenine (A), cytosine (C), thymine (T), and guanine (G).

As used herein, the phrase “plant” includes dicotyledons plants and monocotyledons plants. Examples of dicotyledons plants include tobacco, Arabidopsis, soybean, tomato, papaya, canola, sunflower, cotton, alfalfa, potato, grapevine, pigeon pea, pea, Brassica, chickpea, sugar beet, rapeseed, watermelon, melon, pepper, peanut, pumpkin, radish, spinach, squash, broccoli, cabbage, carrot, cauliflower, celery, Chinese cabbage, cucumber, eggplant, and lettuce. Examples of monocotyledons plants include corn, rice, wheat, sugarcane, barley, rye, sorghum, orchids, bamboo, banana, cattails, lilies, oat, onion, millet, and triticale.

As used herein, the phrase “plant material” refers to leaves, stems, roots, flowers or flower parts, fruits, pollen, egg cells, zygotes, seeds, cuttings, cell or tissue cultures, or any other part or product of a plant. In some embodiment, plant material includes cotyledon and leaf.

A used herein, the phrase “plant tissue” refers to a group of plant cells organized into a structural and functional unit. Any tissue of a plant in planta or in culture is included, for example: whole plants, plant organs, plant seeds, tissue culture and any groups of plant cells organized into structural and/or functional units.

As used herein, the phrases “apparatus” and “device” are used interchangeably unless stated otherwise.

As used herein, the phrase “amino acid” refers to a molecule having the structure wherein a central carbon atom (the alpha (α)-carbon atom, or “Cα”) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino and carboxylic groups in the dehydration reaction that links one amino acid to another. As a result, when incorporated into a protein, an amino acid is referred to as an “amino acid residue.” In the case of naturally occurring proteins, an amino acid residue's R group differentiates the 20 amino acids from which proteins are typically synthesized.

As used herein, the phrase “protein” refers to any polymer of two or more individual amino acids (whether or not naturally occurring) linked via a peptide bond, and occurs when the carboxyl carbon atom of the carboxylic acid group bonded to the α-carbon of one amino acid (or amino acid residue) becomes covalently bound to the amino nitrogen atom of amino group bonded to the α-carbon of an adjacent amino acid. These peptide bond linkages, and the atoms comprising them (i.e., α-carbon atoms, carboxyl carbon atoms (and their substituent oxygen atoms), and amino nitrogen atoms (and their substituent hydrogen atoms)) form the “polypeptide backbone” of the protein. The polypeptide backbone shall be understood to refer the amino nitrogen atoms, α-carbon atoms, and carboxyl carbon atoms of the protein.

Further, the phrase “protein” is understood to include the phrases “polypeptide” and “peptide” (which, at times, may be used interchangeably herein). Molecules comprising multiple polypeptide subunits (e.g., DNA polymerase IE, RNA polymerase II) or other components (for example, an RNA molecule, as occurs in telomerase) are included within the meaning of “protein” as used herein. Fragments of proteins and polypeptides are also within the scope of the invention and may be referred to herein as “proteins.” A protein “domain” refers to mean a portion of a larger protein which, in isolation, assumes a three dimensional conformation corresponding to the conformation the domain assumes when it exists in the larger protein.

As used herein, the phrases “magnetic beads” and magnetic particles” are interchangeable and refer to particles that are intrinsically magnetically responsive or have been rendered magnetic by, for example, attachment to a magnetically responsive substance or by incorporation of such substance into the particles. The magnetic particles can be paramagnetic, ferromagnetic, or superparamagnetic. Exemplary of the magnetic component of particles that are intrinsically magnetic or magnetically responsive include complex salts and oxides, borides, and sulfides of iron, cobalt, nickel and rare earth elements having high magnetic susceptibility, e.g., hematite, ferrite. The magnetic particles may comprise a magnetic component including such metals or alloys.

Typically the magnetic particles may contain a core of the magnetic component with surface functional groups such as hydroxyl, silicate, carboxylate, sulfate, amino, phosphate and the like. Frequently, an additional surface coating will be employed that is covalently or non-covalently bound to the surface. The surface coating can be an anionic or cationic detergent, usually anionic; or the coating can be a protein such as albumin, immunoglobulin, avidin, fetuin or the like; or it can be a carbohydrate such as dextran, chitosan, amylose and the like, or combinations or these substances in their native form or functionalized so as to control their charge and hydrophilicity. Alternatively, the particles can be coated with other amphiphilic substances including lipopolysaccharides or octyl glucoside.

Alternatively, the magnetic component can be incorporated into a particle such as, for example, impregnating the substance in a polymeric matrix. See e.g., Whitesides, et al. (1983) Trends in Biotechnology. 1(5): 144-148, the content of which is herein incorporated by reference in its entirety.

In some embodiments, the barrier-based sample processing apparatus/device use paramagnetic beads to bind the analyte of interest (for example, genomic DNA) for facilitating the rapid purification of nucleic acid and protein. Typically the execution of the assay in the barrier-based sample processing apparatus/devices requires adding liquids to the barrier-based sample processing apparatus/device and moving the paramagnetic beads through the liquids using a magnet.

Provided are drawings to illustrate exemplary embodiments of systems and methods of automating the addition of liquids, which may contain magnetic heads, and the movement of the paramagnetic heads through the liquids in the device using a magnetic force.

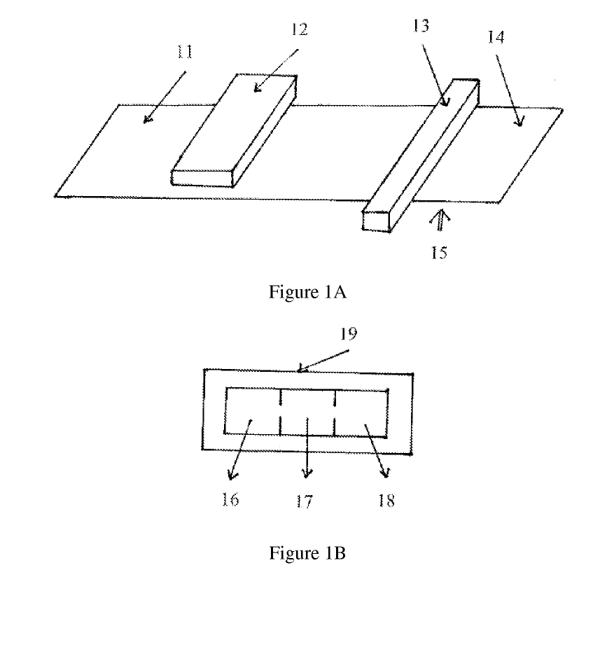

A simple conveyor example is illustrated in FIG. 1. In one aspect, the system comprises a mobile mechanism. In some embodiments, the mobile mechanism comprises a conveyor assembly. The conveyor assembly may include an input area, a liquid transfer station, a magnetic field, and a conveyor. The device(s) may include a sample/oil/reaction liquid device comprising an elution well, an oil well, and a sample well. The sample/oil/reaction liquid device may also be modified to contain an array, for example, having 96 well, 384 wells, or 1536 wells for high-throughput applications. FIG. 1A shows an input area (11), an input station (12) for at least one barrier-based sample processing apparatus/device, a magnetic working area (13) of the automated system provided, where magnetic force or magnetic field (15) can be applied externally. FIG. 1B shows an exemplary embodiment of barrier-based sample processing apparatus (19) with an elution well (16), a barrier well (17), and a sample receiving well (18).

In some embodiments, the assay station is processed as follows:

(1) The sample is loaded on to the conveyor to the sample/oil/reaction liquid transfer station where the appropriate volume of samples is aspirated (sample also contains magnetic beads);

(2) The barrier-based sample processing apparatus/device is loaded onto the conveyor to the sample/oil/reaction liquid input station where the appropriate volume of liquid is dispensed into the sample receiving well of the device;

(3) Elution liquid is dispensed into the elution well of the device;

(4) Oil is dispensed into the well of the device; and

(5) The device is moved via the conveyor over the magnetic field such that the magnetic heads move from the sample well through the oil well into the elution well.

In some embodiments, it is possible that the sample may be transferred using a station that is separate from the additional of the oil and reaction liquid and that the sample container may be off of the conveyor when the sample aliquot is aspirated from it.

In other embodiments, it is possible that there may be multiple input stations for the addition of oil and reaction liquid and there may be multiple oils and reaction liquids. The oil(s) and reaction liquids may be off of the conveyor when the oil or liquid is aspirated by the input station. The conveyor may be of any design including liner and circular.

In further or alternative embodiments, additional samples may be processed by repeating steps 1 through 5 above. The device may have more wells such that more wells will be filled with oil or liquid and the paramagnetic heads will be drawn through the wells that will be positioned in a linear fashion.

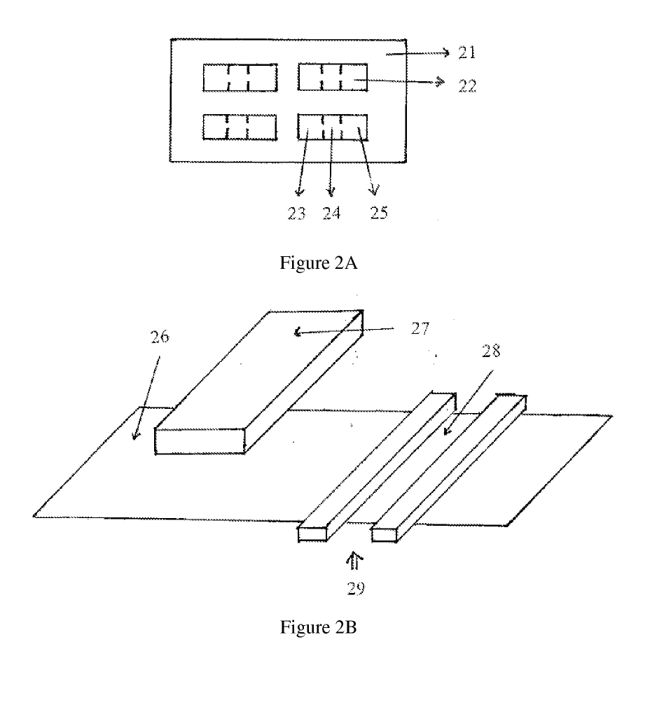

Another exemplary embodiment of the automated systems provided herein with a conveyor is illustrated in FIG. 2, where four barrier-based sample processing apparatuses/devices are located on one plate. FIG. 2A shows an exemplary embodiment of an input station (21) housing four barrier-based sample processing apparatuses/devices (22), where each barrier-based sample processing apparatus/device contains an elution well (23), a barrier well (24), and a sample receiving well (25). FIG. 2B shows an input area (26), a liquid transfer station (27) for at least one barrier-based sample processing apparatus/device, a magnetic working area (28) of the automated system provided, where magnetic force or magnetic field (29) can be applied externally. In some embodiments, there are two magnetic working areas such that all the force can be removed from the magnetic working area and applied to the working area. These embodiments enable simultaneous movement of all paramagnetic particles through the compartments of all of the devices when the forces are applied to the working areas and the devices are moved via the conveyor.

In some embodiments, the barrier-based sample processing apparatuses/devices are processed as follows:

(1) The samples are loaded on to the conveyor in the input area and is moved via the conveyor to the sample/oil/reaction liquid station where the appropriate volumes of samples are aspirated (sample also contains magnetic beads);

(2) The device plate is loaded on to the conveyor in the input area and is moved via the conveyor to the sample/oil/reaction liquid station where the appropriate volume of liquid is dispensed into the right most wells of the devices;

(3) Elution liquid is dispensed into the elute wells of the devices;

(4) Oil is dispensed into the oil wells of the device; and

(5) The plate is moved via the conveyor to the magnetic field station where a magnetic field is applied to the sample wells, from the bottom, and the device is moved via the conveyor such that the beads move from the sample wells to the oil wells to the elution wells in each device.

In some embodiments, it is possible that the samples may be transferred using a station that is separate from the station that adds oil and/or reaction liquid to the device, and that the sample containers may be located off of the conveyor when the sample aliquots are aspirated from the sample container.

In other embodiments, it is possible that there may be multiple input stations for the addition of oil(s) and reaction liquid(s) to the wells. The oil(s) and reaction liquid(s) may be located off of the conveyor when the oil of liquid is aspirated by the input station.

In further or alternative embodiments, additional samples may be processed by repeating steps 1 through 5 above. The conveyor may be any design including linear and circular.

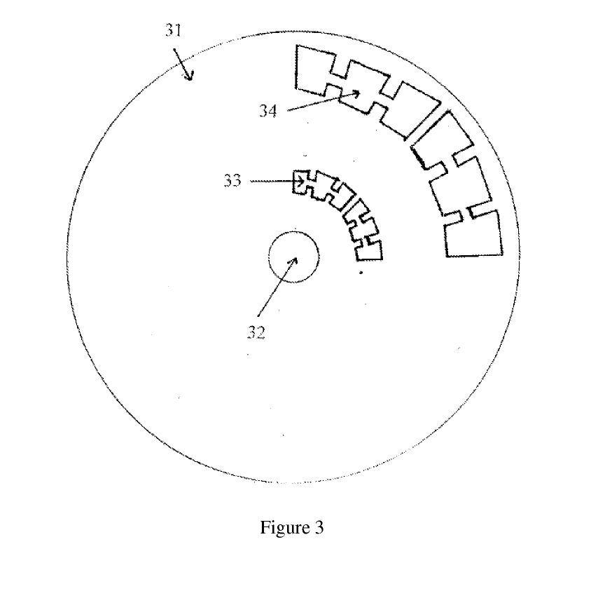

Another exemplary embodiment of the automated systems provided herein with a rotating disk (31) is illustrated in FIG. 3. A rotating anchor axis (32) and multiple barrier-based sample processing apparatuses with different sizes (33 and 34) are shown.

In some embodiments, the barrier-based sample processing apparatus/device is secured on a disk where devices of a shorter length are positioned near to the center of the disk and devices of a longer length are positioned nearer to the outside edge of the disk as shown in FIG. 3. The disk can be partially filled with devices as shown, or completely filled with devices.

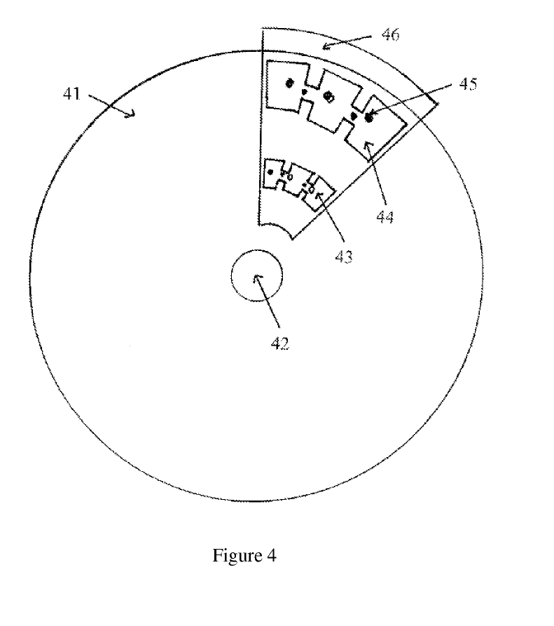

Another exemplary embodiment of the automated systems provided herein with a rotating disk (41) and an aspirating/dispensing station (46) is shown in FIG. 4. A rotating anchor axis (42) and multiple barrier-based sample processing apparatuses with different sizes (43 and 44) are shown. Also shown are multiple dispensing/aspirating nozzles (45) of the aspirating/dispensing station (46).

The automation of adding and removing liquids can be configured in a wedge shaped design such that one wedge shaped portion of the disk can be processed in parallel (at the same time) as shown in FIG. 4. The disk may contain one or more devices. The dispensing head may be configured such that one nozzle dispenses and/or aspirates one liquid or one (each) nozzle dispenses and/or aspirated more than one liquid.

FIG. 5 shows a close view of an exemplary aspirating/dispensing station (51) as part of the automated system shown in FIG. 4. A side wall (52) and two supporting legs (52) of the aspirating/dispensing station (51) are shown.

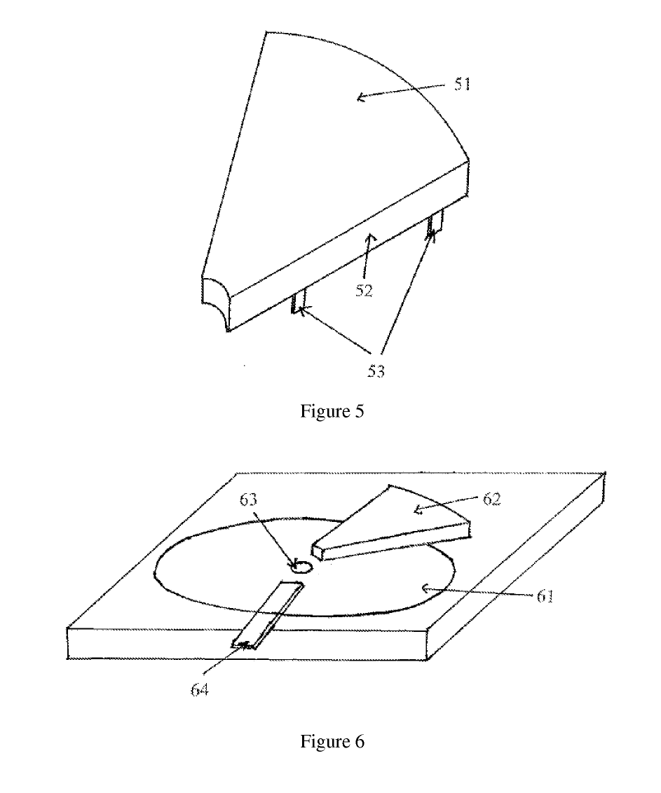

Another exemplary embodiment of the automated systems provided herein with a rotating disk (61) and an aspirating/dispensing station (62) is shown in FIG. 6. A rotating anchor axis (63) and a magnet or magnetic field generator (64) for producing a magnetic force or magnetic field are shown.

The disk can spin to locate the devices under the head and the eluant can be removed when the elution well is over the magnet or after the elution well has been passed over the magnet. One advantage of this configuration is that if samples need to be extracted using several types of magnetic bead and several sizes of devices, the extractions can be performed at the same time point (or approximately the same time).

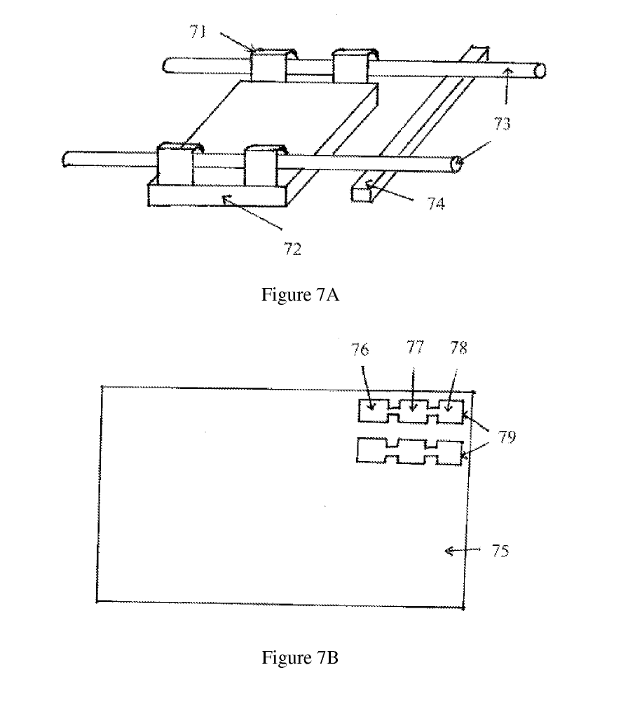

An exemplary embodiment of a movable input station for at least one barrier-based sample processing apparatus/device is shown in FIG. 7. FIG. 7A shows a sled (72) can move along a pair of rails (73) using multiple bushings (71). A magnet or magnetic field generator (74) is also shown. FIG. 7B shows an input station (75) with two barrier-based sample processing apparatuses (79), where each barrier-based sample processing apparatus contains an elution well (76), a barrier well (77), and a sample receiving well (78).

The movement of the magnet is shown in relation to the barrier-based sample processing apparatus/device, or vice versa. Similar to when the device is used in an un-automated setting, the movement of the magnet can be facilitated by a manually operated transfer device. In one embodiment, the magnet can remain stationary while the device or devices move, as illustrated in FIG. 7A.

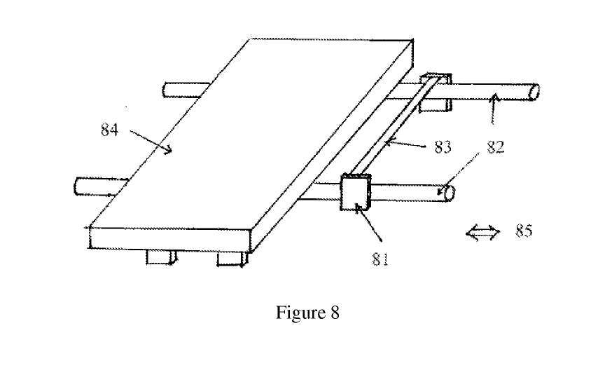

In another embodiment, the magnet moves while the device remains stationary as illustrated in FIG. 8. FIG. 8A shows an exemplary embodiment of a stationary input station, where the magnet or magnetic field generator (83) can move along a pair of rails (82) using a pair of bushings (81) under an input station platform (84). The direction (85) of the movable magnet or magnetic field generator (83) is shown.

One advantage of the manually operated transfer devices described above is that it decreases the possibility of the device being misaligned in the x, y, or z directions which can result in less than optimal movement of the paramagnetic beads through the device or devices.

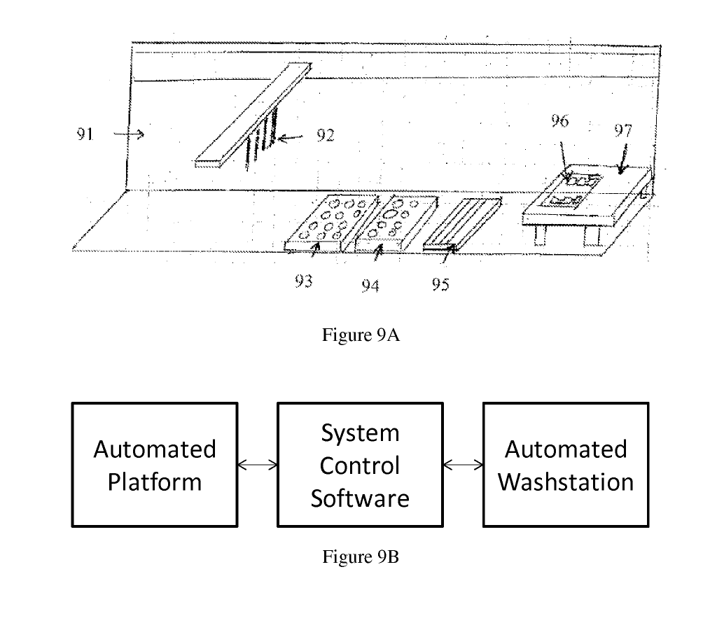

Another exemplary embodiment of the automated system provided herein is shown in FIG. 9. FIG. 9A shows an automated pipetting station or pipetting system with multiple pipette tips (92) for liquid transfer and an automated platform of input station (97) with two barrier-based sample processing apparatuses (96). Also shown are reservoirs (95) for various reagents or solutions, tubes with samples to be processed (94), and tubes with eluted solution from samples that have been process in the system provided (93). FIG. 9B shows relations of various components of the automated system provided herein.

In some embodiments, an automated device which moves the magnet or devices, can be developed by adding an electro mechanical mechanism to move the magnet and/or the device. The electromechanical mechanisms can be triggered via a user interface including computer program or a switch that is mounted on the device. The electro mechanical mechanism can also be triggered or controlled by computer software which also controls other equipment that prepare(s) the samples and solutions into the device.

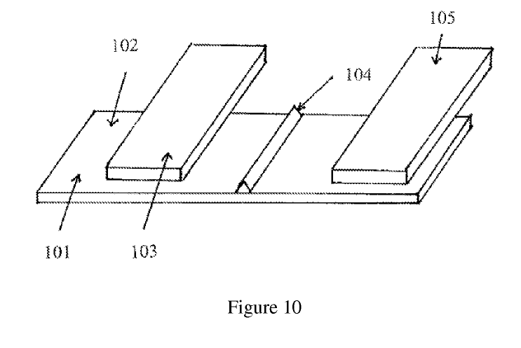

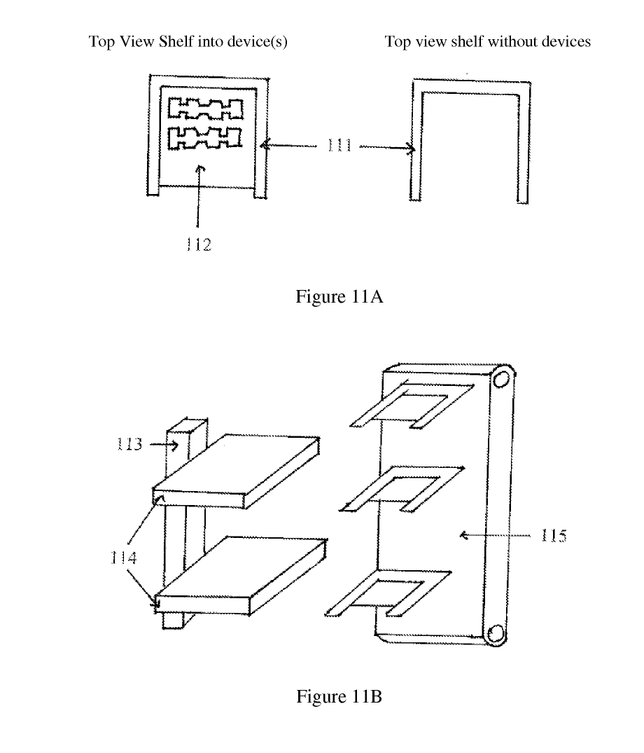

Another exemplary embodiment of the automated system provided herein is shown in FIG. 10. A conveyor (101), an input area (102), a sample process station (103), a magnetic field station (104), and a liquid transfer station (105) are shown (for example FIG. 9B).

In some embodiments, fabricating the devices on one continuous piece of flexible substrate can allow for the use of equipment and methodology as described herein except that the devices need not be placed on or removed from the conveyor belt. In other embodiments, a piece of equipment or methodology to remove the liquid and/or magnetic beads can be added to the methodology described herein and illustrated in FIG. 10. The liquid transfer station may transfer any of the liquids that have been added to the device(s) and transfer the liquids to other vessels. The vessels can be located on the conveyor or off of the conveyor. The liquid transfer station can be above the magnetic fields and/or after the device(s) pass over the magnetic fields.

Another exemplary embodiment of the automated system provided herein with a vertical conveyor is shown in FIG. 11. FIG. 11A shows a shelf (111) linked to the vertical conveyor for housing multiple barrier-based sample processing apparatuses (112; two are shown). FIG. 11B show that multiple shelves are linked to the vertical conveyor (115). Also shown are multiple liquid stations (114) linked to a tower (113) next to the vertical conveyor (115).

In some embodiments, liquid station tower moves horizontally and vertically to access device(s). In other embodiments, a mechanism for automatically moving the magnet under the devices and removing the devices is illustrated in FIG. 11.

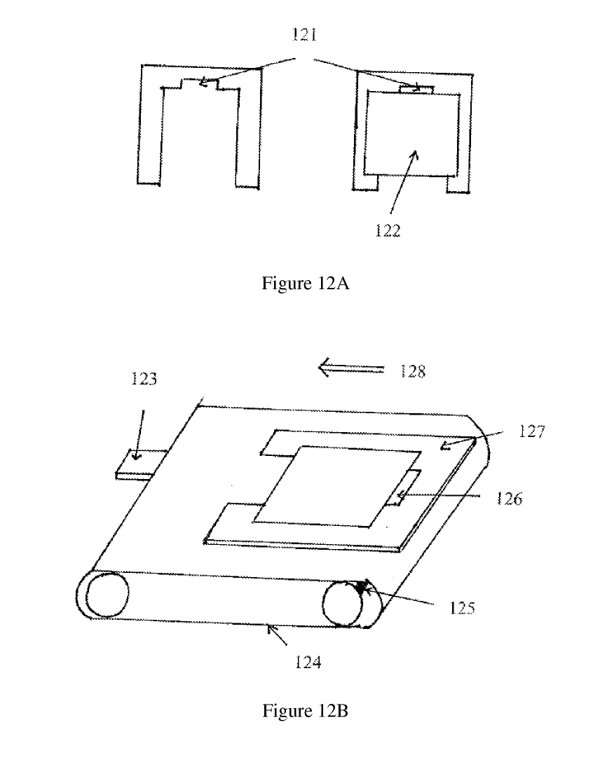

Another exemplary embodiment of the automated system provided herein with a horizontal conveyor is shown in FIG. 12. FIG. 12A shows top view of a shelf without plate (left panel) and with plate (122; right panel), where the plate is removable from the slot (121) of the shelf. FIG. 12B shows a tab (123), a shelf with plate (127), a magnet or magnetic field generator (125), and a belt (124) of the horizontal conveyor. Also shown is the slot (126) of the shelf, and direction of the belt (128).

The mechanism can be positioned under the shelf such that when the belt moves, the magnet moves under the devices on the plate and the magnetic force moves the magnetic beads through the wells in the devices. The belt can then be stepped so that the liquid station is positioned in the plate and removes the liquid(s) from the plates. The liquid station can then be moved away from the plate and shelf. In some embodiments, the belt then moves such that the tab projects through the slot and, as the belt moves, the tab pushes the plate off the shelf.

Although the foregoing invention has been described in some detail by way of illustration and example for the purposes of clarity and understanding, it will be obvious to those skilled in the art that certain changes or modifications may be practiced within the scope of the appended claims.

I claim:

1. An automated system for nucleic acid extraction/isolation from a sample of plant tissue or plant cells, comprising,
(a) at least one barrier-based sample processing apparatus;
(b) a mobility mechanism for transporting the sample processing apparatus;
(c) a plurality of magnetic beads and at least one magnetic field generator;
(d) at least one liquid reagent; and
(e) a mechanism for adding and aspirating the liquid reagent.

2. The automated system of claim 1, further comprising a centrifuge device.

3. The automated system of claim 1, wherein the automated system does not comprise a shaker for mixing solution with the sample processing apparatus.

4. The automated system of claim 1, wherein the at least one barrier-based sample processing apparatus comprises:
(i) an input zone for receiving the sample therein;
(ii) a first reaction zone for receiving a first reagent therein; and
(iii) a second reaction zone downstream of the first reaction zone for receiving a second reagent therein; and
(iv) a force movable between a first position adjacent the input zone and a second position adjacent the second reaction zone; wherein: the force urges the fraction-bound solid phase substrate from the input zone and into the first reaction zone; and the force urges a first portion of the fraction-bound solid phase substrate into the second reaction zone.

5. The automated system of claim 1, wherein the at least one barrier-based sample processing apparatus comprises:
(i) a first chamber for receiving the sample comprising a material of interest;
(ii) a second chamber in communication with the first chamber; and
(iii) a lipophilic or immiscible barrier between the first and second chambers;
wherein upon application of an external force to the material of interest, and positional movement of the force relative to the first chamber and the second chamber effects transfer of the material of interest from the sample in the first chamber, into and through the lipophilic or immiscible barrier, and into the second chamber, wherein the material of interest is extracted from the sample which remains in the first chamber.

6. The automated system of claim 1, wherein the at least one barrier-based sample processing apparatus comprises:
(i) an inlet system provided in an area of the first liquid type;
(ii) a first preparation system provided in an area of the first liquid type; and
(iii) a barrier system provided in an area of the second liquid type;
wherein the inlet system and the first preparation system are separated by the barrier system, and wherein the inlet system is adapted to receive a sample comprising a material of interest and the first preparation system is adapted to receive a receiving liquid.

7. The automated system of claim 1, wherein the at least one barrier-based sample processing apparatus uses a nucleic acid separation method comprising:
(i) allowing nucleic acid in the sample to bind to one or more magnetic beads; and
(ii) passing the nucleic acid-magnetic bead complex through a lipophilic or immiscible barrier to separate the nucleic acid from the sample;
wherein the nucleic acid-bead complex is passed through and separated from the immiscible barrier with an applied magnetic field.

8. The automated system of claim 1, wherein the at least one barrier-based sample processing apparatus uses a nucleic acid isolation method comprising:
(i) contacting the sample with a solid support in the absence of any chaotropic agent, the solid support comprising an organic polymer, whereby nucleic acid in the sample is bound to the support in a sequence-independent manner in the absence of any chaotropic agent, and (ii) separating the support with bound nucleic acid from the sample.

9. The automated system of claim 1, wherein the mobility mechanism comprises a conveyor or a disk.

10. The automated system of claim 9, wherein the mobility mechanism further comprises a robotic arm.

11. The automated system of claim 9, wherein the conveyor comprises a vertical conveyor or a horizontal conveyer.

12. The automated system of claim 1, wherein the magnetic field generator comprises magnetic separator pins or an electromechanical device.

13. The automated system of claim 1, wherein the at least one liquid reagent comprises a nucleic acid binding solution or an elution solution.

14. The automated system of claim 13, wherein the elution solution comprises Tris-HCl at pH 7.0-9.0.

15. The automated system of claim 14, wherein the elution solution comprises Tris-HCl at pH 8.5.

16. The automated system of claim 1, wherein the mechanism for adding and aspirating the liquid reagent comprises a Vprep® pipetting station available from Agilent Technologies Company, or an Evo from Tecan.

17. The automated system of claim 1, wherein the plant is selected from the group consisting of cotton, canola, corn, soybean, sunflower, or wheat.

18. The automated system of claim 1, wherein the plant tissue comprises leaf or cotyledon tissue.

19. The automated system of claim 1, wherein the plant tissue comprises a punch number from 1 to 100.

20. The automated system of claim 1, wherein the throughput of the system is equal or greater than 90 samples per run.

21. The automated system of claim 1, wherein the throughput of the system is equal or greater than 4,000 samples per run.

22. The automated system of claim 1, wherein the throughput of the system is between 200 and 3,000 samples per run.

23. The automated system of claim 12, comprising a plurality of electromechanical or magnetic devices.

24. The automated system of claim 23, wherein the electromechanical device or devices can be turned on or off or can be positioned such that device(s) does or does not affect movement of the magnetic beads.
